(19) United States
(12) Patent Application Publication
(10) Pub. No.: US 2026/0317727 A1
(43) Pub. Date: Oct. 15, 2026

(54) METAL-POLYPHENOL-BASED CELL INACTIVATION COMPOSITION, INACTIVATION METHOD, AND INACTIVATED CELL

(71) Applicant: Novastra Therapeutics Inc., Dover, DE (US)

(72) Inventors: Junling GUO, Suzhou (CN); Yunxiang HE, Suzhou (CN)

(73) Assignee: Novastra Therapeutics Inc., Dover, DE (US)

(21) Appl. No.: 19/555,935

(22) Filed: Mar. 4, 2026

Related U.S. Application Data
(63) Continuation of application No. PCT/CN2025/107831, filed on Jul. 10, 2025.

(30) Foreign Application Priority Data
Apr. 11, 2025 (CN) 202510452177 .3

(51) Int. Cl. C12N 5/00; C12N 1/04; C12N 7/06

(52) U.S. Cl. CPC C12N 5/526; C12N 1/04; C12N 7/06

(57) ABSTRACT

A metal-polyphenol-based cell inactivation composition, method, and resulting inactivated cells are disclosed. The method involves mixing a plant polyphenol solution with a metal ion solution to form a first solution, followed by addition of a first buffer to produce a metal-polyphenol nanoparticle suspension. The first buffer is acidic or neutral. The suspension is then added to a cell suspension containing target cells to obtain a second solution. Subsequently, a second buffer, which is neutral or alkaline and has a higher pH than the first buffer, is introduced to complete cell inactivation. Through coordination between plant polyphenols and metal ions, stable metal-polyphenol nanoparticles are generated. These nanoparticles strongly adhere to the cell surface, forming a dense nano-coating. The coating physically blocks cellular proliferation and metabolic pathways, thereby achieving efficient and stable cell inactivation without disrupting overall structural integrity.

### CROSS-REFERENCE TO RELATED APPLICATIONS

This application is a National Stage Entry of PCT Application No. PCT/CN2025/107831, filed on Jul. 10, 2025, which in turn claims priority to Chinese Patent Application No. 2025104521773, filed on Apr. 11, 2025, the entire contents of each of which are hereby incorporated by reference.

### TECHNICAL FIELD

The present invention relates to the field of biomedical research and application, specifically to a metal-polyphenol-based cell inactivation composition, an inactivation method, and cells inactivated using said method or composition.

### BACKGROUND

Inactivated cells play a critical role in biomedical research and applications. For example, in virus vaccine development (such as influenza vaccines), mammalian cells are inactivated to retain antigenicity, thereby stimulating immune response to defend against live viruses. Similarly, in tumor immunotherapy and vaccine research, inactivated tumor cells serve as an antigen source for the immune system to recognize tumors, inducing an immune response to inhibit tumor growth.

Existing cell inactivation methods include radiation, chemical treatment, physical freezing, and high-temperature treatment. Radiation inactivation utilizes ionizing radiation to destroy the DNA or RNA of tumor cells, causing them to lose reproductive capacity; however, this method may alter cell antigenicity, thereby affecting the immune efficacy. While physical freezing and high-temperature treatments can quickly inactivate cells, they may lead to the destruction of cell structures, which negatively impacts immunogenicity. High-temperature or aggressive chemical treatments often fail to balance inactivation efficiency with antigenic integrity, and residual reagents may trigger systemic inflammatory responses. Chemical drug treatments typically employ chemotherapeutic agents such as cisplatin and doxorubicin; although effective at killing tumor cells, these agents may exhibit toxicity to normal cells and involve complex operational procedures.

Recently, several novel tumor cell inactivation technologies have emerged, such as lactic acid-induced inactivation and the use of specific cell surface anchoring molecules. For instance, Chinese Patent Application No. CN109908334A discloses a method for enhancing the immunogenicity of tumor cells via lactic acid treatment, yet the inactivation efficiency and mildness of this method require further improvement. Chinese Patent Application No. CN116669767A mentions various methods for tumor cell inactivation and immune enhancement, including the use of specific antibody-binding molecules and immune activity enhancers. However, these methods often necessitate complex preparation processes and the coordination of multiple components.

### SUMMARY

It is an object of the present invention to provide a plant-polyphenol-based cell inactivation method. The method forms stable metal-polyphenol (also known as metal-phenolic) nanoparticles through the interaction between plant polyphenols and metal ions. These nanoparticles adhere closely to the surface of the cells to be inactivated, forming a dense nano-coating that physically blocks the proliferation and metabolic pathways of the cells, thereby achieving efficient inactivation. Simultaneously, the method is easy to operate, utilizes mild conditions, effectively enhances experimental efficiency in biomedical research, and improves experimental safety.

The present invention is realized through the following technical solutions:

A metal-polyphenol-based cell inactivation method, comprising the following steps:
mixing a plant polyphenol solution and a metal ion solution to obtain a first solution;
adding a first buffer to the first solution to obtain a metal-polyphenol particle suspension;
adding the metal-polyphenol particle suspension to a cell suspension of cells to be inactivated to obtain a second solution;
adding a second buffer to the second solution to inactivate the cells to be inactivated in the second solution;
wherein the first buffer is an acidic buffer or a neutral buffer, the second buffer is a neutral buffer or an alkaline buffer, and a pH value of the first buffer is lower than a pH value of the second buffer.

In the present technical solution, the plant polyphenol solution and the metal ion solution are first mixed to obtain the first solution. In some preferred embodiments, the plant polyphenol solution is first dispersed in physiological saline, followed by the addition of the metal ion solution and uniform mixing to obtain the first solution. In one or more embodiments, the plant polyphenol solution is obtained by dissolving plant polyphenols in a solvent, and the metal ion solution is obtained by dissolving metal salts in a solvent, wherein the solvent is preferably deionized water.

After obtaining the uniformly mixed first solution, the pH value of the first solution is adjusted by adding the first buffer to obtain a metal-polyphenol particle suspension. The first buffer may be an acidic or neutral buffer, and in some preferred embodiments, the pH value of the first buffer is 4-6.5. In one or more embodiments, the first buffer may be at least one of an acetate buffer, a citrate buffer, a phosphate buffer, and a succinate buffer. More preferably, the first buffer is a phosphate buffer with a pH of 5.0-6.0. Utilizing the abundant phenolic hydroxyl groups in plant polyphenols, metal-polyphenol nanoparticles can be formed through coordination with metal ions. By regulating the pH via the first buffer, the size of the nanoparticles formed by metal-polyphenol complexation can be controlled to 5-50 nanometers.

The metal-polyphenol particle suspension is added to the cell suspension of the cells to be inactivated to obtain a second solution, followed by the addition of the second buffer to the second solution to inactivate the cells. The second buffer is a neutral or alkaline buffer, and in some preferred embodiments, the pH value of the second buffer is 7-9. In one or more embodiments, the second buffer may be at least one of a phosphate buffer, a Tris buffer, and a borate buffer. Further preferably, the second buffer is a phosphate buffer with a pH of 8.0-9.0. After the small metal-polyphenol nanoparticles adhere to the cell surface, the pH of the second solution is increased via the higher-pH buffer system, causing the nanoparticles to link tightly and form a dense network, thereby physically blocking cell proliferation and metabolism to ultimately inactivate the cells.

Two pH adjustments are performed in the present technical solution. During the first adjustment, the lower pH results in incomplete complexation between the plant polyphenols and metal ions, forming nanoparticles of very small size with weak inter-particle connections. Once these small nanoparticles bind to the bacterial surface, the second adjustment increases the pH, promoting tighter connections between the nanoparticles and between the particles and the cells. This forms a dense network with extremely small gaps on the cell surface, effectively blocking proliferation and metabolic pathways to achieve cell inactivation.

Furthermore, in some embodiments, a stabilizer such as a hyaluronic acid solution may be added to the first solution to improve the stability of the metal-polyphenol nanoparticles. This forms an even denser nano-coating on the cell surface, which not only facilitates inactivation but also effectively prevents the leakage of intracellular contents, maintains structural integrity, and reduces risks associated with content leakage. For example, coating stem cells can prevent the leakage of key contents such as TGF-β and IGF-1 during in vitro manipulation and transplantation, thereby maintaining the therapeutic potential of the stem cells.

Further, a molar ratio of metal ions in the metal ion solution to plant polyphenols in the plant polyphenol solution is 4:1 to 1:3.

The content of metal ions affects the size and stability of the nanoparticles. If the metal ion content is too low, it is difficult to form stable nanoparticles or coatings, leading to decreased inactivation efficiency. Conversely, if the metal ion content is too high, stability increases but the particle size also increases, creating larger gaps in the nano-coating and likewise reducing the inactivation effect. Therefore, the molar ratio is preferably 4:1 to 1:3.

Further, the concentration of the plant polyphenol solution is 100-400 mg/mL, and the concentration of the metal ion solution is 40-250 mg/L.

In some embodiments, the plant polyphenol solution concentration is preferably 150-250 mg/mL, and the metal ion solution concentration is preferably 40-150 mg/L.

Further, the cells to be inactivated are resuspended in physiological saline, concentrated by centrifugation, and then resuspended in physiological saline to obtain the cell suspension.

In some embodiments, pre-treated tumor cells are resuspended in 1 mL of physiological saline (0.9% NaCl) to a concentration of 1×10⁵ to 1×10⁷ cells/mL. The suspension is concentrated by centrifugation (200-800 rcf, 3-5 min), and the cells are resuspended in 50-400 μL of physiological saline.

Further, the plant polyphenol is at least one selected from the group consisting of: proanthocyanidin (PC), tara tannin (Tara), bayberry tannin (BT), black wattle tannin (BWT), larch tannin (LT), tannic acid (TA), ellagic acid (EA), epigallocatechin gallate (EGCG), catechin gallate (CG), and catechin.

Further, the metal ion in the metal ion solution is at least one selected from the group consisting of: zinc ions, manganese ions, iron ions, and aluminum ions.

The present invention further provides an inactivated cell obtained by inactivating cells via any of the aforementioned metal-polyphenol-based methods, wherein the surface of the inactivated cell is coated with a metal-polyphenol nanolayer composed of metal-polyphenol particles.

The inactivated cells may be of various types, such as tumor cells or bacterial cells, providing efficient and mild inactivated cells for tumor immunotherapy or influenza vaccine development. The cells may also be stem cells that retain key contents post-inactivation to maintain therapeutic potential, or virus cells where the nano-coating reduces endotoxin leakage to improve safety. Thus, the inactivation method provided herein has broad applicability and versatility.

Additionally, this cell surface modification strategy based on metal-polyphenol nanostructures is dynamic and modular, providing significant advantages for applications requiring adaptability. It serves as a modular, cell-independent surface functionalization method, offering new directions for cell engineering.

The present invention further provides a metal-polyphenol-based cell inactivation composition, comprising a metal ion solution, a plant polyphenol solution, a first buffer, and a second buffer. The metal ion solution and plant polyphenol solution are mixed to form a first solution; the first buffer adjusts the pH of the first solution to acidic or neutral to form a metal-polyphenol particle suspension; and the second buffer adjusts the pH of the cell suspension containing the particles to neutral or alkaline to form a metal-polyphenol nanolayer on the cell surface to inactivate the cells.

The components of the composition may be used in any of the aforementioned inactivation methods. In some embodiments, the four reagents are preferably independently packaged, though the metal ion and plant polyphenol solutions, or those two plus the first buffer, may be pre-mixed and packaged together.

Further, the pH of the first buffer is 4.0-6.5, and the pH of the second buffer is 7.0-9.0.

Compared with the prior art, the present invention has the following advantages and beneficial effects:
1. Efficient inactivation is achieved by forming a stable, dense metal-polyphenol nano-coating on the cell surface to physically block proliferation and metabolic pathways.
2. The process is simplified, requiring no complex equipment and involving only straightforward steps like solution preparation and mixing, which reduces cost, operational difficulty, and risks associated with complex procedures. 3. The two-step pH regulation ensures the formation of small, loosely connected particles that subsequently link into an extremely dense network upon binding to the cell surface, effectively blocking metabolism. 4. The method maintains structural integrity and prevents the leakage of intracellular contents, reducing safety risks. 5. Optimized molar ratios between metal ions and polyphenols facilitate the formation of stable, smaller-sized nanoparticles on the cell surface.

### BRIEF DESCRIPTION OF THE DRAWINGS

The drawings described herein are intended to provide a further understanding of the embodiments of the present invention and constitute a part of this application, but do not limit the embodiments of the present invention. In the drawings:

FIG. 1 is a flow chart of a cell inactivation method according to an embodiment of the present invention;

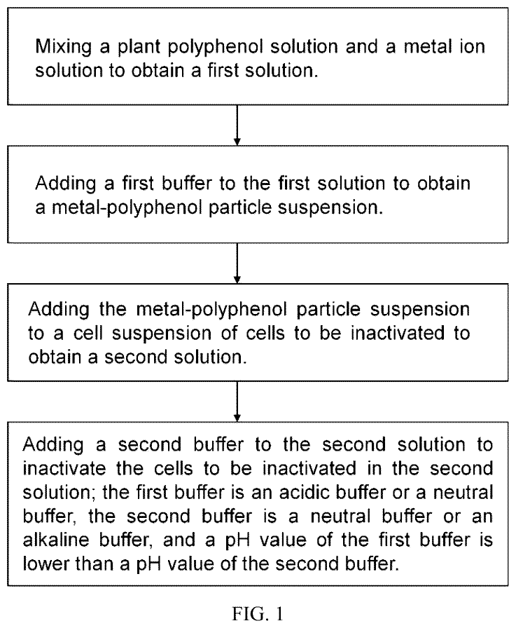

FIG. 2 illustrates transmission electron microscopy (TEM) characterization of the surfaces of an untreated tumor cell and an inactivated tumor cell C1 according to an embodiment of the present invention;

FIG. 3 illustrates the surface polyphenols of a tumor cell C1 labeled with fluorescein-bovine serum albumin (FITC-BSA) according to an embodiment of the present invention;

FIG. 4 illustrates transmission electron microscopy (TEM) characterization of the surfaces of an untreated bacterial cell and an inactivated bacterial cell C6 according to an embodiment of the present invention;

FIG. 5 illustrates the surface polyphenols of a bacterial cell C6 labeled with fluorescein-bovine serum albumin (FITC-BSA) according to an embodiment of the present invention;

FIG. 6 illustrates a comparison of tumor cells before and after inactivation treatment using Calcein/PI live-dead cell staining according to an embodiment of the present invention;

FIG. 7 illustrates cell viability statistics of untreated tumor cells and tumor cells C1 at different time points according to an embodiment of the present invention;

FIG. 8 illustrates the cell survival rates of tumor cells C1 to C5 according to an embodiment of the present invention;

FIG. 9 illustrates the surface potential characterization of tumor cells C1 to C5 according to an embodiment of the present invention;

FIG. 10 illustrates the survival rate of bacterial cells C6 before and after inactivation according to an embodiment of the present invention;

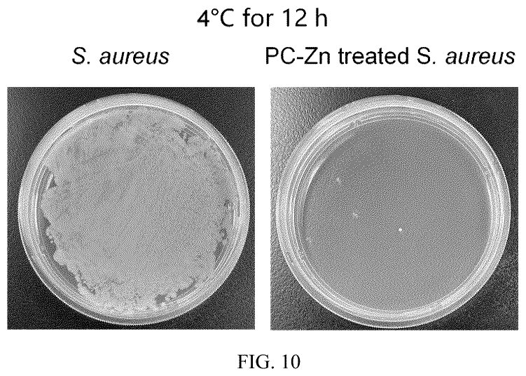

FIG. 11 illustrates electrophoresis results of sediments from untreated stem cells and stem cells C7 after rupture according to an embodiment of the present invention;

FIG. 12 illustrates the protein content and RNA content collected from stem cells C7 after rupture according to an embodiment of the present invention.

### DETAILED DESCRIPTION OF THE EMBODIMENTS

To make the objectives, technical solutions, and advantages of the present invention clearer, the present invention is further described in detail below with reference to the embodiments and accompanying drawings. The exemplary embodiments and descriptions thereof are intended to explain the present invention and should not be construed as limiting the scope of the invention.

All raw materials used in the present invention have no particular restriction on their sources and may be purchased commercially or prepared according to conventional methods well known to those skilled in the art. There is no particular restriction on the purity of the raw materials; analytical grade purity or purity requirements conventional in the field of cell inactivation are preferred. The grades and abbreviations of all raw materials are conventional in the art; each grade and abbreviation is clear and definite within its relevant field of use, and those skilled in the art can purchase them commercially or prepare them through conventional methods based on the grade, abbreviation, and corresponding use.

The terms “first”, “second”, and the like (e.g., first solution and second solution, first phosphate buffer and second phosphate buffer, etc.) are used for clarity of description to distinguish corresponding components and are not intended to limit any order or emphasize importance. Furthermore, unless otherwise specified, the term “connected” as used herein may refer to a direct connection or an indirect connection via other groups.

### I. Preparation of Inactivated Cells

Currently, metal-polyphenol particles are primarily used for cell delivery, such as probiotic delivery, which requires maintaining the activity and function of the cells during the delivery process. Therefore, in the preparation of conventional metal-polyphenol particles, the polyphenols are first attached to the cells, and then metal ions are combined with the polyphenols to form a nano-coating. Such nano-coatings have very large gaps, through which the cells realize their functions.

In the inactivation method of the present invention, to form a fine and dense metal-polyphenol particle coating on the cell surface, loose and small metal-polyphenol nanoparticles are first formed in a low-pH environment. After these are connected to the cells, the pH is adjusted to a high value to form a dense network on the cell surface, which physically blocks the proliferation and metabolic pathways of the cells, thereby achieving the purpose of inactivating the cells in the second solution.

The metal-polyphenol-based cell inactivation method employed in the present embodiments comprises the following steps:
mixing a plant polyphenol solution and a metal ion solution to obtain a first solution;
adding a first buffer to the first solution to obtain a metal-polyphenol particle suspension;
adding the metal-polyphenol particle suspension to a cell suspension of cells to be inactivated to obtain a second solution; and
adding a second buffer to the second solution to inactivate the cells to be inactivated in the second solution;
wherein the first buffer is an acidic buffer or a neutral buffer, the second buffer is a neutral buffer or an alkaline buffer, and the pH value of the first buffer is lower than the pH value of the second buffer.

Specifically, Examples 1 to 7 illustrate preferred embodiments of inactivating cells by forming a nano-coating on the cell surface using plant polyphenols and metal ions.

### Example 1

This example prepares inactivated tumor cells encapsulated in a PC-Zn nano-coating:
S1: Proanthocyanidin (PC) solution (200 μL, 150 mg/mL) was dispersed in 1 mL of physiological saline, and then zinc chloride (ZnCl₂) solution (40 μL, 150 mg/mL) was added; the mixture was shaken uniformly on a shaker (100 rpm, 5 min) to obtain a first solution.
S2: A first phosphate buffer (pH=5.0, 20 mM, 1 mL) was added to the first solution and shaken uniformly (100 rpm, 5 min) to obtain a PC-Zn nanoparticle suspension.
S3: Pre-treated B16F10 tumor cells (2×10⁵ cells) were resuspended in physiological saline (0.9% NaCl solution, 1 mL); 400 μL of the PC-Zn nanoparticle suspension was added to the cell suspension and shaken uniformly (200 rpm, 5 min) to obtain a second solution.
S4: A second phosphate buffer (pH=8, 20 mM, 500 μL) was added to the second solution and shaken uniformly (200 rpm, 5 min) to obtain inactivated tumor cells C1 encapsulated in the PC-Zn nano-coating.

### Example 2

This example prepares inactivated tumor cells encapsulated in an EGCG-Mn nano-coating:
S1: Epigallocatechin gallate (EGCG) solution (400 μL, 200 mg/mL) was dispersed in 1 mL of physiological saline, and then manganese chloride (MnCl₂) solution (80 μL, 100 mg/mL) was added and shaken uniformly (100 rpm, 5 min) to obtain a first solution.
S2: A first phosphate buffer (pH=5.0, 20 mM, 1 mL) was added to the first solution and shaken uniformly (100 rpm, 5 min) to obtain an EGCG-Mn nanoparticle suspension.
S3: Pre-treated B16F10 tumor cells (2×10⁵ cells) were resuspended in 1 mL of physiological saline; 400 μL of the EGCG-Mn nanoparticle suspension was added and shaken uniformly (200 rpm, 5 min) to obtain a second solution.
S4: A second phosphate buffer (pH=8, 20 mM, 500 μL) was added and shaken uniformly (200 rpm, 5 min) to obtain inactivated tumor cells C2 encapsulated in the EGCG-Mn nano-coating.

### Example 3

This example prepares inactivated tumor cells encapsulated in a TA-Fe nano-coating:
S1: Tannic acid (TA) solution (100 μL, 250 mg/mL) was dispersed in 1 mL of physiological saline, then ferric chloride (FeCl₃) solution (50 μL, 100 mg/mL) was added and shaken uniformly (100 rpm, 5 min) to obtain a first solution.
S2: A first phosphate buffer (pH=5.0, 20 mM, 1 mL) was added and shaken uniformly (100 rpm, 5 min) to obtain a TA-Fe nanoparticle suspension.
S3: Pre-treated B16F10 tumor cells (2×10⁵ cells) were resuspended in 1 mL of physiological saline; 400 μL of the TA-Fe nanoparticle suspension was added and shaken uniformly (200 rpm, 5 min) to obtain a second solution.
S4: A second phosphate buffer (pH=8, 20 mM, 500 μL) was added and shaken uniformly (200 rpm, 5 min) to obtain inactivated tumor cells C3 encapsulated in the TA-Fe nano-coating.

### Example 4

This example prepares tumor cells inactivated by the plant polyphenol Tara tannin (Tara) alone:
S1: Tara solution (300 μL, 250 mg/mL) was dispersed in 1 mL of physiological saline, then a first phosphate buffer (pH=5.0, 20 mM, 1 mL) was added and shaken uniformly (100 rpm, 5 min) to obtain a Tara nanoparticle suspension.
S2: Pre-treated B16F10 tumor cells (2×10⁵ cells) were resuspended in 1 mL of physiological saline, and 400 μL of the Tara nanoparticle suspension was added and shaken uniformly (200 rpm, 5 min).
S3: A second phosphate buffer (pH=8, 20 mM, 500 μL) was added and shaken uniformly (200 rpm, 5 min) to obtain inactivated tumor cells C4 encapsulated in a Tara nano-coating.

### Example 5

This example prepares tumor cells modified only with manganese ions (Mn²⁺):
S1: MnCl₂ solution (300 μL, 200 mg/mL) was dispersed in 1 mL of physiological saline, then a first phosphate buffer (pH=5.0, 20 mM, 1 mL) was added and shaken uniformly (100 rpm, 5 min) to obtain a Mn²⁺/Mn(OH)₂ nanoparticle suspension.
S2: Pre-treated B16F10 tumor cells (2×10⁵ cells) were resuspended in 1 mL of physiological saline; 400 μL of the Mn²⁺/Mn(OH)₂ nanoparticle suspension was added and shaken uniformly (200 rpm, 5 min).
S3: A second phosphate buffer (pH=8, 20 mM, 500 μL) was added and shaken uniformly (200 rpm, 5 min) to obtain tumor cells C5 treated with Mn²⁺/Mn(OH)₂ nanoparticles.

### Example 6

This example prepares inactivated bacterial cells encapsulated in a PC-Zn nano-coating:
S1: PC solution (200 μL, 150 mg/mL) was dispersed in 1 mL of physiological saline, then ZnCl₂ solution (40 μL, 150 mg/mL) was added and shaken uniformly (100 rpm, 5 min) to obtain a first solution.
S2: A first phosphate buffer (pH=5.0, 20 mM, 1 mL) was added and shaken uniformly (100 rpm, 5 min) to obtain a PC-Zn nanoparticle suspension.
S3: Pre-treated bacterial cells (S. aureus, 1×10⁷ cells/mL, OD600≈0.1) were resuspended in 1 mL of physiological saline; 400 μL of the PC-Zn nanoparticle suspension was added and shaken uniformly (200 rpm, 5 min) to obtain a second solution.
S4: A second phosphate buffer (pH=8, 20 mM, 500 μL) was added and shaken uniformly (200 rpm, 5 min) to obtain inactivated bacterial cells C6 encapsulated in the PC-Zn nano-coating.

### Example 7

This example prepares inactivated stem cells encapsulated in an EGCG-Mn nano-coating:
S1: EGCG solution (200 μL, 150 mg/mL) was dispersed in 1 mL of physiological saline, then MnCl₂ solution (40 μL, 150 mg/mL) was added and shaken uniformly (100 rpm, 5 min) to obtain a first solution.
S2: Hyaluronic acid (HA) solution (1 mg/mL) and a first phosphate buffer (pH=5.0, 20 mM, 1 mL) were added to the first solution and shaken uniformly (100 rpm, 5 min) to obtain an EGCG-Mn nanoparticle suspension.
S3: Pre-treated stem cells (2×10⁵ cells) were resuspended in 1 mL of physiological saline; 400 μL of the EGCG-Mn nanoparticle suspension was added and shaken uniformly (200 rpm, 5 min) to obtain a second solution.
S4: A second phosphate buffer (pH=8, 20 mM, 500 μL) was added and shaken uniformly (200 rpm, 5 min) to obtain stem cells C7 encapsulated in a coating composed of HA-embedded EGCG-Mn nanoparticles.

In some embodiments, the concentration range of the plant polyphenol solution (e.g., PC or Tara solution) may be 100-400 mg/mL, and is preferably 150-250 mg/mL. The plant polyphenol solution is obtained by dissolving polyphenols in a solvent, which is preferably deionized water.

In some embodiments, the concentration range of the metal ion solution may be 40-250 mg/L, and is preferably 40-150 mg/L. The metal ion solution is obtained by dissolving metal salts in a solvent, which is preferably deionized water.

In one or more embodiments, allogeneic tumor cells are collected and pre-treated by: collecting tumor cells in the logarithmic growth phase; removing serum from the medium via centrifugation (800-1000 rpm, 3-5 min); and washing the cells 1-2 times with serum-free medium or PBS to ensure a clean cell surface for subsequent processing.

In one or more embodiments, autologous tumor cells are collected and pre-treated by: isolating autologous tumor cells using enzymatic digestion, mechanical shearing, or manual tissue grinding to process tumor tissue into separated autologous tumor cells; and washing the cells 1-2 times with serum-free medium or PBS to ensure a clean cell surface.

In one or more embodiments, bacterial cells are collected and pre-treated by collecting bacterial cells and suspending them in physiological saline or phosphate buffer for subsequent processing.

In some embodiments, mammalian cells (e.g., tumor cells, immune cells, erythrocytes) are collected after inactivation by: centrifugation (200-800 rcf, 3-5 min) following the reaction; washing the inactivated cells 3-5 times with phosphate buffer (pH=7-9, 5-100 mM, 600-1500 μL); resuspending the cells in 1 mL of physiological saline; and allowing the cells to stand at 4-30 °C for 8-24 hours to achieve efficient and mild inactivation.

In some embodiments, bacterial cells (E. coli, S. aureus, S. epidermidis, S. pyogenes, P. aeruginosa, etc.) are collected after inactivation by: centrifugation (2000-4000 rcf, 3-5 min) following the reaction; washing the inactivated bacterial cells 3-5 times with phosphate buffer (pH=7-9, 5-100 mM, 600-1500 μL); resuspending the bacterial cells in 1 mL of physiological saline; and allowing the cells to stand at 4-30 °C for 8-24 hours.

In some embodiments, the inactivated cells are resuspended in a suitable buffer for subsequent use in vaccine research, preparation, or other immunotherapy applications.

### II. Characterization and Performance Testing of Inactivated Cells

### Example 8

This example characterizes the inactivated tumor cells C1 encapsulated in the PC-Zn nano-coating.

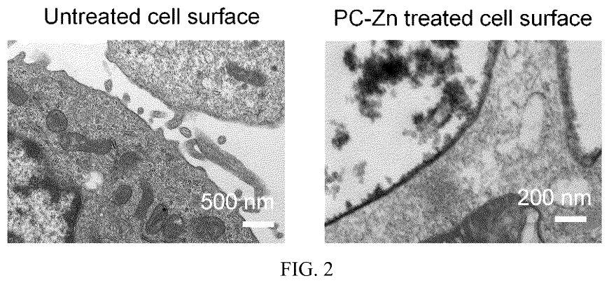

Specifically, transmission electron microscopy (TEM) was used to characterize the cell surface. FIG. 2 shows TEM images of a cross-section of tumor cells C1 and original tumor cells. As shown, a dense nano-coating is formed on the surface of tumor cells C1, whereas the original tumor cells exhibit a smooth cell membrane. This indicates that tumor cells C1 are completely encapsulated by the PC-Zn nano-coating formed from PC and Zn ions.

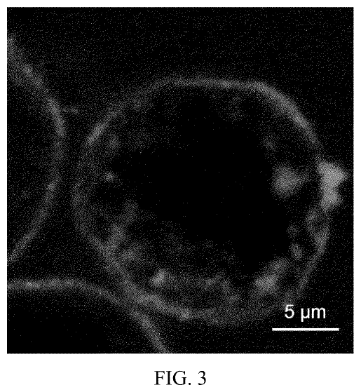

Further, after modification with PC-Zn, a FITC-BSA fluorescent probe was added. As shown in FIG. 3, fluorescently labeled polyphenols demonstrate the loading of PC-Zn on the cell surface, confirming the presence of the PC-Zn nano-coating.

### Example 9

This example characterizes the inactivated bacterial cells C6 encapsulated in the PC-Zn nano-coating.

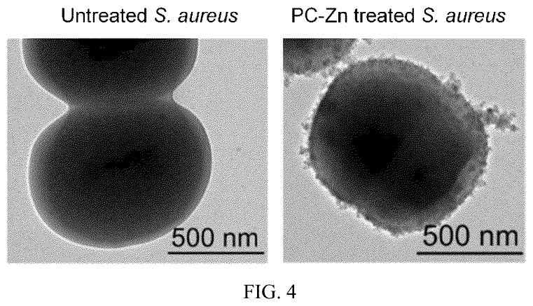

TEM was used to characterize the surface of bacterial cells (S. aureus). FIG. 4 shows TEM images of bacterial cells C6 and original bacterial cells. A dense nano-coating is formed on the surface of bacterial cells treated with PC-Zn, whereas original bacterial cells exhibit a smooth cell membrane, indicating complete encapsulation.

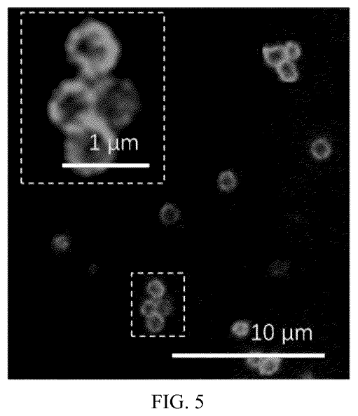

Further, after modification with PC-Zn, a FITC-BSA probe was added. Confocal microscopy results in FIG. 5 show fluorescence around the cells, indicating the presence of PC-Zn nanoparticles on the cell surface.

### Example 10

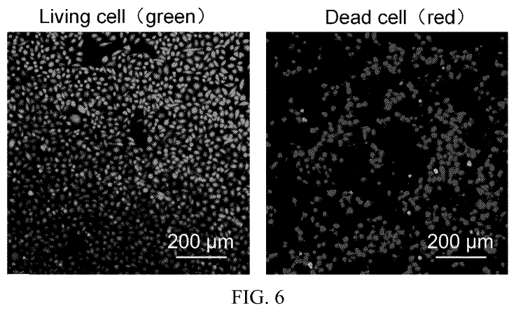

This example detects cell activity after inactivation using Calcein/PI staining. Tumor cells C1 were incubated in DMEM medium (10% serum, 1% antibiotics) in a cell incubator (37 °C, 5% CO₂ for up to 12 hours. Cells were collected, washed with PBS, and treated with 195 μL Annexin V-FITC and 5 μL PI (per 5×10⁴ cells). After incubation in the dark at room temperature for 20 min, detection was performed via laser confocal microscopy. FIG. 6 shows that tumor cells C1 completely lose proliferation capacity within 12 hours, with cell viability near zero, demonstrating significant inactivation.

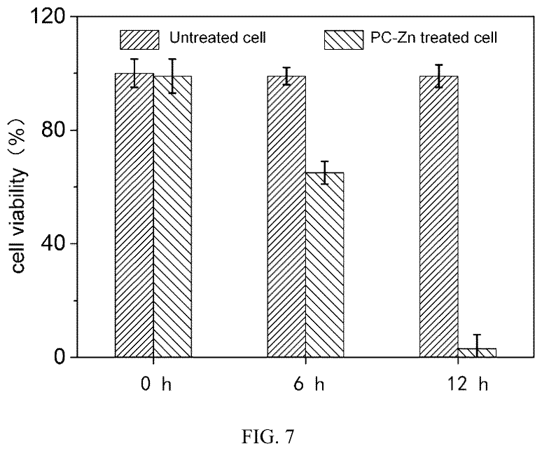

The activity of tumor cells C1 was also measured via CCK-8 assay. As shown in FIG. 7, within 12 hours, tumor cells C1 completely lose proliferation capacity with near-zero activity.

### Example 11

The activity of tumor cells C1-C5 was detected via CCK-8 assay. Cells were incubated in opaque white 96-well plates for 12 hours. 10 μL of CCK-8 solution was added per well and incubated for 1 hour, then absorbance at 450 nm was detected using a microplate reader (Infinite 200PRO, Tecan). Original B16F10 tumor cells served as a control group.

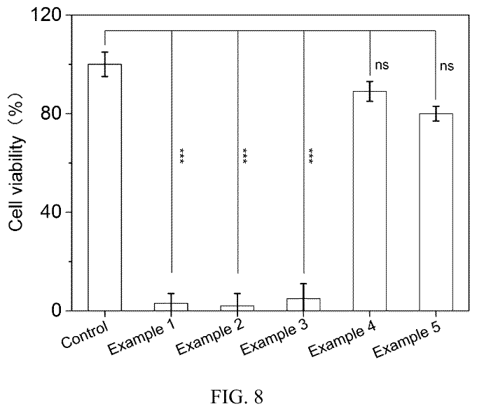

As shown in FIG. 8, the survival rates of tumor cells C1, C2, and C3 inactivated by metal-polyphenol nano-coatings were nearly zero. In contrast, the inactivation effect of cells C4 and C5 (modified only with polyphenols or metal) was far inferior, indicating that the synergistic effect of plant polyphenols and metal ions forms a denser nano-coating that physically blocks proliferation and metabolism.

### Example 12

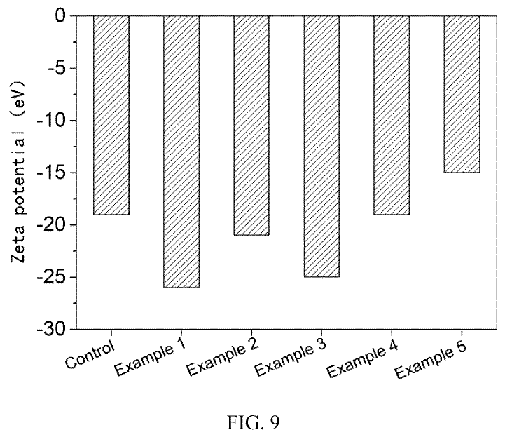

Zeta potentials of tumor cells C1-C5 were measured. As shown in FIG. 9, the surface charge of original tumor cells shifted in a more negative direction after encapsulation by the metal-polyphenol nano-coating, due to the negative charge of plant polyphenols. Tumor cells C1-C3 exhibited different potential values based on the polyphenol and metal ion composition. Potentials for cells C4 and C5 did not change significantly, indicating that individual metal or polyphenol coatings cannot stably encapsulate cells.

### Example 13

Bacterial cell activity was measured via plate counting. After 12 hours of treatment with metal-polyphenol nanomaterials, the activity of bacterial cells C6 was zero, demonstrating significant inactivation.

### Example 14

This example compares the content of intracellular components after lysis between untreated stem cells and stem cells C7 modified with EGCG-Mn.

Equal amounts of untreated stem cells and EGCG-Mn-treated stem cells C7 were ruptured via hypotonic treatment, centrifuged at 300 g for 3 min, and the supernatant and sediment were separated.

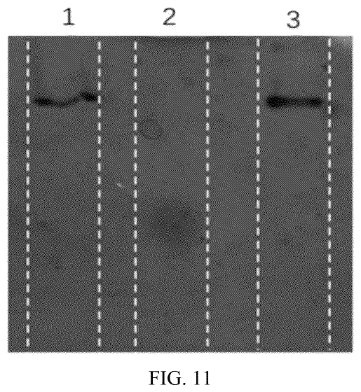

Protein content in the sediment was analyzed via gel electrophoresis. In FIG. 11, lane 1 is the protein profile of control stem cells, lane 2 is from untreated stem cells, and lane 3 is from inactivated stem cells C7 with the EGCG-Mn coating.

The protein content collected from stem cells inactivated by the present method was substantially the same as the original cells, indicating the nano-coating effectively retains intracellular contents (e.g., TGF-β, IGF-1). For example, in myocardial infarction treatment, retaining the vascular growth factor VEGF secreted by stem cells can significantly enhance angiogenesis and treatment success.

Proteins were extracted using RIPA lysis buffer containing protease inhibitors, and concentration was determined via BCA assay. RNA was extracted using Triquick reagent and quantified via NanoDrop 2000.

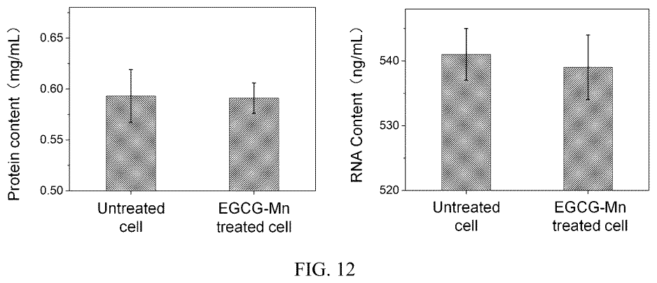

FIG. 12 shows the protein and RNA content in the sediment of stem cells C7 compared to control stem cells. The nano-coating effectively prevented leakage, retaining a large amount of RNA and nearly all protein.

The specific embodiments described above further detail the objectives, technical solutions, and beneficial effects of the present invention. It should be understood that these are merely specific embodiments and are not intended to limit the scope of the invention. Any modifications, equivalent substitutions, or improvements made within the spirit and principles of the present invention shall fall within the scope of protection of the present invention.

What is claimed is:

1. A metal-polyphenol-based cell inactivation method, comprising:
mixing a plant polyphenol solution and a metal ion solution to obtain a first solution;
adding a first buffer to the first solution to obtain a metal-polyphenol particle suspension;
adding the metal-polyphenol particle suspension to a cell suspension of cells to be inactivated to obtain a second solution; and
adding a second buffer to the second solution to inactivate the cells in the second solution;
wherein the first buffer is an acidic buffer or a neutral buffer, the second buffer is a neutral buffer or an alkaline buffer, and a pH value of the first buffer is lower than a pH value of the second buffer.

2. The method according to claim 1, wherein the pH value of the first buffer is 4-6.5, and the pH value of the second buffer is 7-9.

3. The method according to claim 1, wherein a molar ratio of metal ions in the metal ion solution to plant polyphenols in the plant polyphenol solution is 4:1 to 1:3.

4. The method according to claim 1, wherein the plant polyphenol solution has a concentration of 100-400 mg/mL, and the metal ion solution has a concentration of 40-250 mg/L.

5. The method according to claim 1, wherein the cell suspension is obtained by resuspending the cells to be inactivated in physiological saline, concentrating the cells via centrifugation, and then resuspending the centrifuged cells in physiological saline.

6. The method according to any one of claim 1, wherein the plant polyphenol in the plant polyphenol solution is at least one selected from the group consisting of: proanthocyanidins, tara tannin, bayberry tannin, black wattle tannin, larch tannin, tannic acid, ellagic acid, epigallocatechin gallate, catechin gallate, and catechin.

7. The method according to any one of claim 1, wherein metal ions in the metal ion solution are at least one selected from the group consisting of: zinc ions, manganese ions, iron ions, and aluminum ions.

8. An inactivated cell produced by the method according to any one of claim 1, wherein a surface of the inactivated cell is coated with a metal-polyphenol nanolayer composed of metal-polyphenol particles.

9. A metal-polyphenol-based cell inactivation composition, comprising: a metal ion solution, a plant polyphenol solution, a first buffer, and a second buffer;
wherein the metal ion solution and the plant polyphenol solution form a first solution upon mixing;
the first buffer is configured to adjust the pH of the first solution to acidic or neutral to form a metal-polyphenol particle suspension; and
the second buffer is configured to adjust the pH of a cell suspension containing the metal-polyphenol particle suspension to neutral or alkaline to form a metal-polyphenol nanolayer on a surface of cells to be inactivated to inactivate the cells.

10. The composition according to claim 9, wherein the pH value of the first buffer is 4.0-6.5, and the pH value of the second buffer is 7.0-9.0.
